(19) United States
(12) Patent Application Publication
(10) Pub. No.: US 2009/0227848 A1
(43) Pub. Date: Sep. 10, 2009

(54) PROCEDURE AND DEVICE FOR THE SYNCHRONIZATION OF A PHYSIOLOGICAL STATE OF AN INDIVIDUAL WITH A DESIRED STATE

(76) Inventor: Xavier Lefebvre, Nancy (FR)

Correspondence Address: FLEIT GIBBONS GUTMAN BONGINI & BIANCO P.L., ONE BOCA COMMERCE CENTER, 551 NORTHWEST 77TH STREET, SUITE 111, BOCA RATON, FL, 33487, US

(21) Appl. No.: 11/908,056

(22) PCT Filed: Feb. 22, 2006
(86) PCT No.: PCT/EP2006/060184

§ 371 Date: Sep. 29, 2008

(30) Foreign Application Priority Data
Mar. 7, 2005 (FR) FR 05 02237

(51) Int. Cl. A61B 5/00

(52) U.S. Cl. 600/301

(57) ABSTRACT

The invention relates to a procedure and an associated device to bring an individual from an initial physiological state (EP0) to a final physiological state (EPf). The physiological state of the individual is defined by a set of physiological parameters (RP1A, RP1B, RP1C, RP1D, RP2A, RP2B, RP3A, RP3B, RP3C, . . . ), which are characteristic of one or more physiological rhythms of the individual. The set of physiological parameters comprises at least two physiological parameters among a value (RP1A), a variability (RP1B), an amplitude of the variations (RP1C), and a periodicity of the variations (RP1D) of a physiological rhythm (RP1) of the individual.

The invention relates to a procedure for the synchronization of a physiological state of an individual with a desired physiological state that is associated with a desired psychological state.

It is well known that an individual is more or less sensitive to external stress depending on his/her psychological state. Experience thus shows that an unfocused individual has difficulty registering what a third party tells them, for example, in the context of a discussion, training, etc. In an entirely different context, but in the same area of thought, it has also been shown that a stressed individual does not tolerate pain as well as a relaxed individual.

The psychological state of an individual (fear, excitation, stress, joy, relaxation, concentration, etc.) at a given time is favored notably by his/her physiological state at that given time. The physiological state of an individual is defined by his/her physiological rhythms (respiratory rhythm, cardiac rhythm, etc.) and their evolution. The same physiological state can promote several psychological states, and a variation in a particular physiological rhythm can favor one psychological state over another.

Thus, several psychological states can be associated with the same physiological state. However, the reverse is also conceivable, although, for the same psychological state, the physiological characteristics of different persons, or even of the same person in situations that are different but generate the same psychological state, present similar characteristics.

A procedure described in the document WO0051677 detects, evaluates and displays the variations and the variability (i.e., the regularity of the amplitudes of variations) of the frequency of the cardiac rhythm of an individual and his/her state of relaxation. The individual can then act to vary his/her state of relaxation. This procedure is limited to a detection and an interpretation of the physiological rhythms, and it is up to the individual to exploit the information supplied.

Moreover, Huygens demonstrated that two mechanical pendulums of slightly different frequencies synchronize when they are positioned in the vicinity of each other. On the other hand, if the frequencies of the pendulums are too different from each other, the pendulums do not synchronize. This concept of Huygens has been applied to guide an individual to a physiological state associated with a desired target psychological state (easing of tension, relaxation, etc.). For this purpose, an individual is exposed to the emission of a stimulus at an appropriate frequency to cause the physiological rhythms of the individual to change to target physiological rhythms.

Such a procedure is described notably in the document U.S. Pat. No. 5,267,942. The procedure described in this document is limited to changing the frequency of the physiological rhythm considered, which alone is insufficient to guide an individual to the desired psychological state.

A principal purpose of the invention is to improve the procedure described in the document U.S. Pat. No. 5,267,942 by proposing a more effective procedure that makes it possible to cause the physiological rhythms of the individual to change more precisely, more rapidly, and in a manner that is totally transparent to the individual, to the target physiological rhythms associated with the desired psychological state, in such a way as to facilitate the individual's attainment of this desired psychological state.

Thus, the invention concerns a procedure to bring an individual from an initial physiological state to a final physiological state, where the physiological state of the individual is defined by a set of physiological parameters that are characteristic for one or more physiological rhythms of the individual. The set of physiological parameters comprises at least two physiological parameters among a value, a variability, an amplitude of the variations, and a periodicity of the variations of a physiological rhythm of the individual. The procedure according to the invention comprises the following steps:
E2: determination of the initial physiological state of the individual,
E3: exposure of the individual for a first period to a set of stimuli, comprising at least one stimulus, where said stimulus is characterized by at least one stimulation parameter whose value is a function of the initial physiological state,
E4: determination of an intermediate physiological state between the initial physiological state and the final physiological state,
E5: modification of at least one parameter of at least one stimulus of the set of stimuli as a function of the intermediate state, an exposure of the individual to the modified set of stimuli,
Q8: if the intermediate physiological state is different from the desired final physiological state, repetition of steps E4 and E5.

The procedure is improved by the addition of the following steps, which are carried out between steps E5 and Q8:
E6: determination of the physiological state reached by the individual,
Q7: if the physiological state reached by the individual is different from the intermediate physiological state, repetition of steps E5 and E6, and by the following step Q9, which is carried out between steps E6 and E7:
Q9: if the physiological state reached by the individual is further removed from the intermediate physiological state than the physiological state reached previously by the individual and determined during step E2 or during a preceding step E6, repetition of steps E3 and following while choosing a new intermediate physiological state that is closer to the physiological state reached by the individual.

The following step E10 may also be carried out, before step E3:
E10: selection of a set of stimuli, as a function of the initial physiological state of the individual, and of the final physiological state and optionally following step Q11, which is carried out after step E6:
Q11: if the set of stimuli is not effective or of low effectiveness, carrying out step E10, and then steps E3 and following.

Finally, the procedure can also comprise a step of initialization comprising the following substeps, during which:
E0: a physiological state is associated with each desired psychological state in a table, where each physiological state is characterized by a set of physiological parameters,
E1: a final physiological state is selected from the table as a function of a desired psychological state.

The invention also concerns a device intended for the production of stimuli intended to lead an individual from an initial physiological state to a final physiological state, comprising:
a means of measuring the current physiological state of an individual,
a means of analyzing and comparing the measured physiological state with a reference physiological state, which is either an intermediate physiological or a final physiological state to be reached,
a means of determining the intermediate physiological state, as a function of the determined current physiological state of the individual and of the final physiological state to be reached,
a means of producing a set of stimuli corresponding to at least one stimulus of which at least one stimulation parameter is variable as a function of the determined intermediate state, and
a means of exposing the individual to the produced set of stimuli.

It is preferred for the physiological state of the individual to be defined by a set of physiological parameters that are characteristic of one or more physiological rhythms of the individual, where the set of physiological parameters comprises at least two physiological parameters, and the analyzing and comparing means comprises:
a means of analyzing the measured current physiological state, to extract from it the physiological parameters,
a means of comparing the extracted physiological parameters with corresponding parameters of the reference physiological state.

The set of physiological parameters comprises preferably at least two parameters among a value, a variability, an amplitude of the variations, and a periodicity of the variations of a physiological rhythm of the individual.

The measurement means comprises, for example, a sensor that measures cardiac rhythm, respiratory rhythm and/or brain waves of the individual.

The device according to the invention can also comprise a calibration means, to test several sets of stimuli and to classify the tested sets of stimuli as a function of the effectiveness on the physiological state of the individual, and optionally a means of selecting an effective set of stimuli as a function of the current physiological state of the individual.

Thus, in the context of the invention, the overall physiological state of the individual is considered, taking into account at least two physiological parameters that are characteristic of one or more physiological rhythms of the individual. The two parameters considered can be selected among a value, a variability, an amplitude of the variations, and a periodicity of the variations of a physiological rhythm of the individual. The selection of the number of parameters and of the type of parameters for each physiological rhythm is notably a function of the relevance of the parameters of each rhythm.

It should be noted that the purpose of the invention is simply to influence the physiological rhythms of one or more individuals: after having determined the physiological state of an individual, one or more appropriate stimuli are emitted in the direction of the receptive organs of the individual to guide him/her to a target physiological state.

However, inducing artificially in an individual the physiological state associated with a certain psychological state does not mean that the individual will access this psychological state. Thus, the objective of the invention is not so much to hope to induce a desired psychological state (fear, excitation, stress, joy, relaxation, concentration, . . . ) in an individual but to induce the associated physiological state in such a way as to increase the probability of the individual changing from the initial psychological state to the target psychological state.

For example, it is known that a nervous person does not memorize efficiently. In this case, the goal is to bring the physiological state of the individual to a physiological state that is associated with a psychological state of concentration. The path that remains to be traveled for the individual to learn better is reduced accordingly. The individual will then have to induce the additional spark that allows him/her to make the step to an effective concentration.

Other characteristics and advantages of the invention will become clear in the following description of a preferred embodiment of a device and a procedure according to the invention. This description is for information and is in no way limiting, and it is made in reference to the drawings in the appendix, in which:

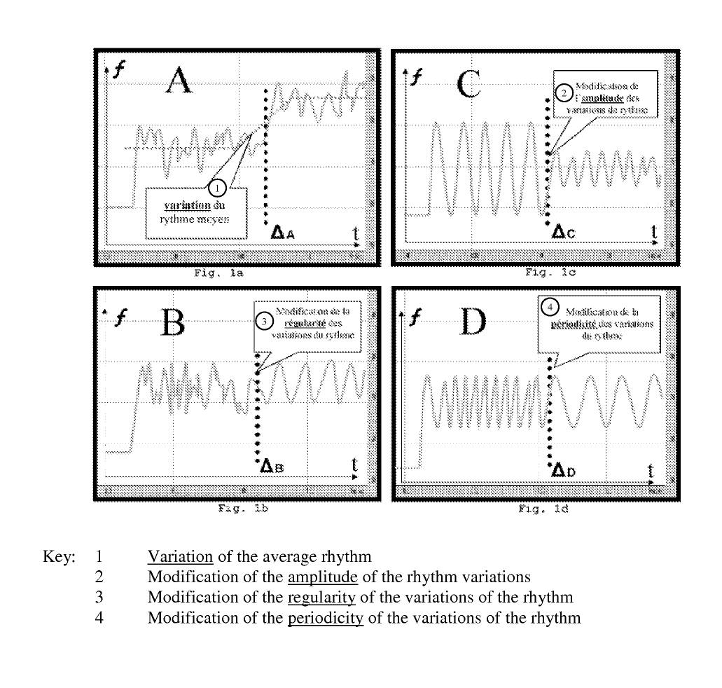

FIGS. 1a-1d show four parameters associated generally with a physiological rhythm of an individual,

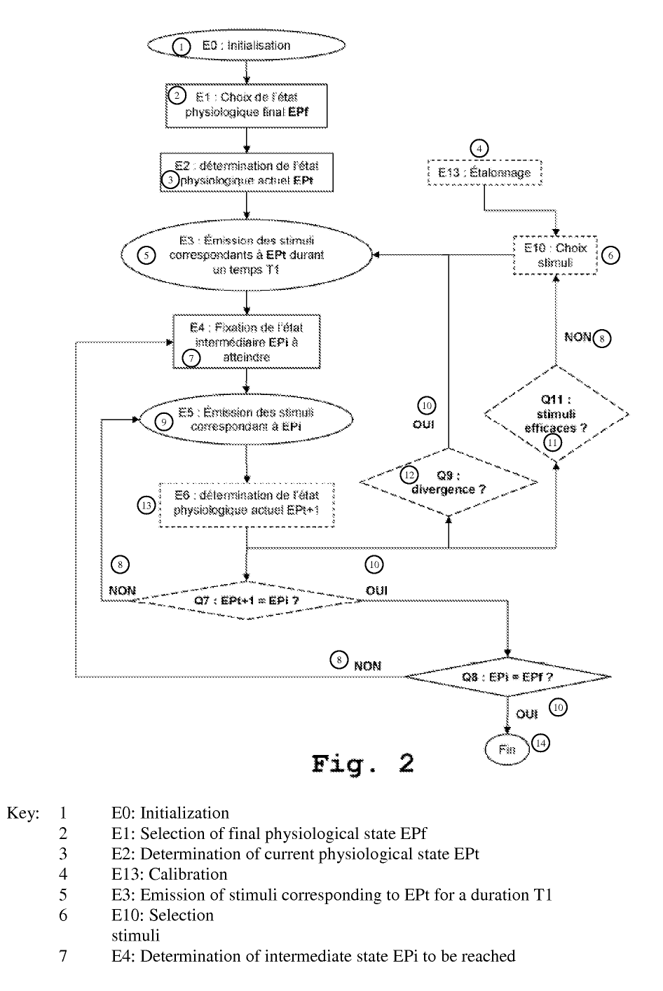

FIG. 2 is a flow chart of a procedure according to the invention, and

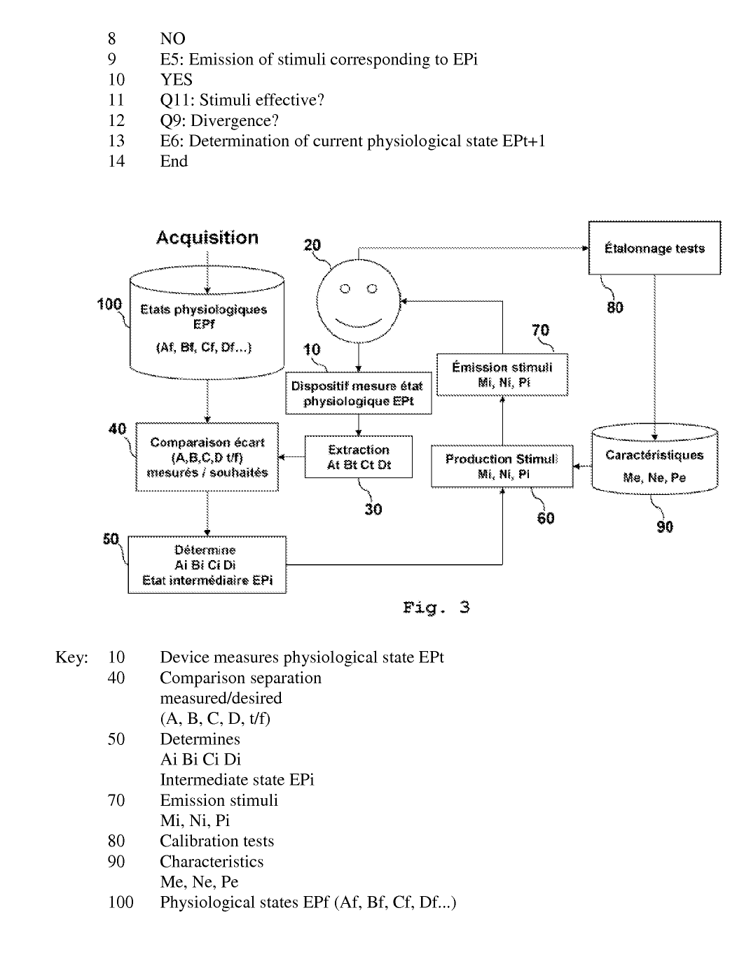

FIG. 3 is a block diagram of an appropriate device for carrying out the procedure according to the invention.

### DEFINITIONS

An individual is considered here who is in an initial physiological state EP0, who wishes to come to a final physiological state EPf. For example, the state EP0 is associated with a psychological state of stress, and the state EPf is associated with a psychological state of concentration. At time t, the physiological state of the individual is EPt.

The physiological state EPt of the individual is defined by a set of physiological rhythms RP1, RP2, RP3 . . . . For example, RP1 is the cardiac rhythm, RP2 is the respiratory rhythm, . . . of the individual.

Each physiological rhythm is defined by a number (optionally variable) of parameters. For example, the rhythm RP1 is defined by four parameters RP1A, RP1B, RP1C, RP1D; the rhythm RP2 is defined by two parameters RP2A, RP2B; the RP3 by three parameters RP3B, RP3C, etc. RP1A, RP1B, RP1C, RP1D are, in an example, the value, the variability, the amplitude of the variations, and the periodicity of the variations of the cardiac rhythm RP1, respectively.

The physiological state of the individual can thus be defined by a set of physiological parameters (RP1A, RP1B, RP1C, RP1D, RP2A, RP2B, RP3A, RP3B, RP3C, . . . ) regrouping all the parameters of all the physiological rhythms considered.

Moreover, to act on the physiological state EP of the individual, a set is used comprising one or more stimuli ST1, ST2, ST3, etc. For example, ST1 is an audio signal, such as background music; ST2 is a video signal, such as an image projected on a screen; ST3 is a tactile signal, such as an armchair whose resting surface is set in motion, etc.

Each stimulus is defined by a number (optionally variable) of parameters. For example, the stimulus ST1 is defined by three parameters ST1M, ST1N, ST1P, the stimulus ST2 is defined by the parameter ST2P; the stimulus ST3 is defined by two parameters ST2N, ST2P. For example, in the audio signal ST1, the parameters ST1M, ST1N, ST1 are the sound level, the frequency, and the level of harmonic 1 with respect to the fundamental component of the signal, respectively.

Each set of stimuli (ST1, ST2, ST3, . . . ) is thus defined by a set of stimulation parameters (ST1M, ST1N, ST1P, ST2P, ST3N, ST3P, etc. . . . ).

Finally, with each set of physiological set of parameters (RP1A, RP1B, RP1C, RP1D, RP2A, RP2B, RP3A, RP3B, RP3C, . . . ) of the physiological state EP, a set of stimulation parameters (ST1M, ST1N, ST1P, ST2P, ST3N, ST3P, etc. . . . ) of the set of stimuli is associated, such that, when the individual is exposed to the set of stimuli so defined:
his/her physiological state remains unchanged, if it is identical to the associated physiological state, or
his/her physiological state evolves to the associated physiological state, if it is different.

### Evolution of Physiological Rhythms

Experience has shown that all human physiological rhythms (cardiac, respiratory rhythms, brain waves, etc.) are signals whose frequency evolves in a similar way as a function of time regardless of the rhythm considered. Each rhythm can then be characterized by several parameters, which are obtained by applying a temporal mathematical processing or fast Fourier transform (FFT) processing to the frequency signal as a function of time. For example, the four parameters represented in FIGS. 1a to 1d are considered:
the value of the average frequency (FIG. 1a),
the variability of the frequency, i.e., the regularity of the variations of the average frequency (FIG. 1b),
the amplitude of the variations of the frequency (FIG. 1c), and
the periodicity of the variations of the frequency (FIG. 1d).

To simplify, these parameters are called A, B, C, D, respectively, below.

It should be noted that, depending on the physiological rhythm considered, one can take into account only one, two or three parameters among those described above. Parameters other than the four parameters described above may be selected, particularly if they are more relevant. The selection of the number of parameters defining a physiological rhythm is in practice a compromise between the complexity of implementation and the effectiveness of the procedure, which increase naturally with the number of parameters.

The curves pertaining to the frequency of the rhythm and to its parameters have similar shapes regardless of the physiological rhythm considered. Only the values of the parameters change, in more or less large proportions, as a function of the physiological rhythm considered, of the individual considered, and of his/her psychological state.

Experience thus shows, for example, that, for a group of individuals in a psychological state, the value of the average frequency of the same physiological rhythm varies considerably from one individual to the other. For example, for several individuals in a state of relaxation, the average value A of the frequency of their cardiac rhythm (i.e., cardiac pulsation) varies considerably from one individual to the other. On the other hand, provided that the psychological state of the individuals of the group does not change, the average frequency varies very little over time for the same individual, and the variability, the amplitude of the variations, and the periodicity of the variations of the average frequency vary very little from one individual to the other. It is thus advantageous to take these last parameters B, C, D into account in the context of the invention.

### Description of an Embodiment of the Procedure of the Invention (FIG. 2)

The description below of embodiment examples of the procedure and of the device of the invention is provided for the following simplified case. The physiological state EPt of the individual is considered here at a time t to be defined by its cardiac rhythm RP1 alone, where the latter is defined by four parameters RP1A, RP1B, RP1C, RP1D. Moreover, to act on the physiological state of the individual, a set of stimuli is used comprising a single stimulus ST1, of the audio type, comprising three parameters ST1M, ST1N, ST1P, respectively the frequency, the overall level, and the level of the harmonics with respect to the fundamental signal of the audio signal.

A procedure according to the invention comprises essentially the following steps.

First is measured the initial physiological state EP0 of an individual (step E2) and the physiological parameters (RP1A, RP1B, RP1C, RP1D) are determined corresponding to this EP0.

This is followed by production of a set of stimuli, associated with the state EP0 for a duration T1 (step E3), whose destination is the perception organs of the individual (in the simplified example, the ear). This step makes it possible to “lock onto” the individual, i.e., to synchronize the physiological state EP0 of the individual with the produced set of stimuli.

In the example, an audio signal is produced with the parameters (ST1M, ST1N, ST1P), which are associated with the parameters (RP1A, RP1B, RP1C, RP1D) of the cardiac rhythm of the individual.

Then, an intermediate physiological state EPi desired for the individual to reach is determined (step E4). The intermediate step is between the current physiological state of the individual and a final physiological state EPf desired for the individual to reach. Selection of the intermediate state is preferably made taking into consideration the following points:
the selected intermediate state EPi must be closer to the final state EPf than the current state, since it is desired to approach the final state, and
the intermediate state EPi must be close enough to the current state of the individual so that Huygens' principle can be applied, i.e., so that the actual physiological state of the individual does indeed come closer to the intermediate physiological state EPi.

The intermediate step EPi and the final state EPf, are selected, for example, from a table listing the different possible physiological states. Each physiological state is defined by a set of parameters (RP1A, RP1B, RP1C, RP1D) or more generally (RP1A, RP1B, RP1C, RP1D, RP2A, RP2B, RP3A, RP3B, RP3C, . . . ), and each physiological state is associated with a possible psychological state.

Then, a stimulus (here an audio signal) is produced having parameters (ST1M, ST1N, ST1P) which correspond to the parameters (RP1Ai, RP1Bi, RP1Ci, RP1Di) associated with the physiological state EPi. The stimulus is produced for a duration T2 (step E5). The duration T2 is selected to be sufficiently long so that the parameters of the cardiac rhythm of the individual who perceives the stimulus reach the values (RP1Ai, RP1Bi, RP1Ci, RP1Di).

Then, steps E4 and E5 are repeated if the intermediate step EPi is different from the final state EPf (step E8). Two physiological states are identical if their respective physiological parameters are identical. Steps E4 and E5 are thus repeated once or several times to bring the individual to the final state EPf, little by little passing through successive intermediate steps EP1, EP2, . . . , EPi, EPi+1, EPi+2, . . . until the state EPf is reached. The selection of the successive states and of the number of successive states is performed as a function of the difference between the measured initial state EP0 of the individual and the desired final state EPf.

Improvements that can be Made to the Above-Described Procedure

### Selection of the Temporal Parameters T1, T2:

The durations T1 and/or T2 can be modified as a function of the individual on which the procedure is carried out. This can be done, for example, during a step of initialization E0 of the procedure.

### Continuous Measurement of the Physiological State of the Individual and Variation in Real Time of the Parameter T2:

The duration of a reaction to a stimulus can be highly variable from one individual to another, or for the same individual at different times. Consequently, the duration T2 must be selected to be sufficiently long to allow the individual exposed to the stimulus time to react satisfactorily. Under these conditions, it may sometimes take unnecessarily long to carry out the procedure, for example, for individuals who react relatively rapidly to a stimulus.

To overcome this drawback, it is possible to measure continuously (or at time intervals T3 that are much smaller than T2) the current physiological state EPt of the individual (step E6). If the parameters (RP1At, RP1Bt, RP1Ct, RP1Dt) of the measured physiological state EPt are equal to the parameters (RP1Ai, RP1Bi, RP1Ci, RP1Di) of the intermediate state EPi (step Q7), then step E5 is stopped. Otherwise, steps E5, E6 are repeated.

### Continuous Measurement of the Physiological State of the Individual and Resynchronization if Necessary:

It is possible for an individual to react poorly to a produced set of stimuli, in the sense that, when he/she is exposed to the set of stimuli, the actual physiological state EPt of the individual moves away from instead of closer to the desired physiological state (the intermediate state EPi or the final state EPf, depending on the case). This may be the case, for example, if the selected desired physiological state is too far removed from the initial state of the individual. In this case, the individual cannot reach the desired state.

To overcome this disadvantage, it is possible to measure continuously (or at short time intervals T3) the physiological state EPt of the individual (step E6) and, if the measured state EPt comes close to the desired state (step Q9), the procedure continues, otherwise, steps E3 and following are repeated, while choosing a new, more appropriate intermediate state EPi, which is closer to the actual state of the individual.

### Selection of a Set of Stimuli Among Several:

Several types of stimuli can be used in the context of the procedure to cause the physiological rhythms of an individual change. For example, stimuli of the audio type may be used (music, words, special sounds, etc.), or the visual type (images, text, as displayed on a screen, a panel, etc.), or the tactile type (deformation of an object held by the individual, etc.).

Also, for the same stimulus, different parameters can be varied (for example, the contours and the color of an object in an image can be varied simultaneously). A combination of different stimuli may also be used.

In practice, one individual may be more sensitive to a given set of stimuli than another; an individual may also be more sensitive than another to the same variation of the same parameter of the same stimulus of the set of stimuli to which he/she is exposed. Also, the same individual, at different times, may present different sensitivities to the same stimulus.

The procedure can also be improved by allowing the selection of the most appropriate set of stimuli. The selection can be made, depending on the case, among other possibilities as a function of:
the initial state EP0 of the exposed individual,
the final state EPf to be reached,
the separation between the initial state EP0 of the individual and the final state EPf to be reached,
the desired maximum time for the individual to reach the final state EPf,
selected intermediate states.

In these cases, the stimulus or the stimuli can be selected at the beginning of the procedure, for example, during a step E10 that is carried out before step E5.

The selection of the stimulus or of the stimuli can also be modified once or several times during the procedure, for example, as a function of the reactions of the individual stimulated by the selected stimulus or stimuli. For this purpose, a step E11 can be provided, during which the effectiveness of the stimulus used is determined, then, if the stimulus is effective, the procedure continues, or otherwise a more effective type of stimulus (step E10) is selected, and steps E5 and following of the procedure are repeated.

A first set of stimuli is considered to be more effective than a second set of stimuli if, when the individual is exposed to the first set of stimuli for the purpose of reaching a physiological state EPi associated with a first set of stimuli, the physiological state of the individual more rapidly approaches the state EPi than if he/she were exposed to the second set of stimuli.

A step of calibration of the procedure (step E13) can also be carried out, for example:
at the beginning of the procedure,
during a first utilization of the procedure by a given individual, or optionally
continuously throughout the entire utilization of the procedure.

Calibration has the purpose of determining, for a given individual, at a given time, the effectiveness of the different types of stimuli, and, optionally, of determining, for each stimulus, the effectiveness of a variation of each parameter of the stimulus.

### Description of an Embodiment of a Device According to the Invention

A device according to the invention comprises essentially (FIG. 3):
a measurement device 10 capable of measuring the evolution of the physiological state EPt of an individual 20 over time,
a means 30 of analyzing the measured physiological state EPt, to extract from it the physiological parameter(s) (RP1At, RP1Bt, RP1Ct, RP1Dt, RP2At, RP2Bt, RP3At, RP3Bt, RP3Ct, . . . ),
a means 40 of comparing the extracted physiological parameter(s) (RP1At, RP1Bt, RP1Ct, RP1Dt, RP2At, RP2Bt, RP3At, RP3Bt, RP3Ct, . . . ) with one or more corresponding parameters (RP1A, RP1B, RP1C, RP1D, RP2A, RP2B, RP3A, RP3B, RP3C, . . . ) of a desired physiological state (EPi or EPf, depending on the case),
a means 50 of determining the physiological parameters (RP1Ai, RP1Bi, RP1Ci, RP1D1, RP2A1, RP2B1, RP3A1, RP3B1, RP3Ci, . . . ) of an intermediate physiological state EPi between the physiological state EPt of the individual as measured and an expected final physiological rhythm EPf,
a means 60 of producing a set of stimuli associated with the intermediate physiological rhythm EPi, and
means 70 of emitting the stimulus or the stimuli whose destination is the perception organs of the individual, to stimulate the individual.

The measurement device 10 comprises at least one sensor for measuring a physiological rhythm of an individual. For example, known measurement sensors may be used that are optionally adapted to be preferably nonintrusive and as imperceptible as possible to the individual. In an example, the sensor 10 measures the cardiac rhythm of the individual; it may be, for example, an arterial pressure sensor, such as those known in medicine. The device 10 is used essentially to carry out steps E2 and E6.

The means 30 is a mathematical processing of a temporal signal, to extract from it, by a calculation of mean values and/or fast Fourier transforms (FFT), the parameters of each physiological rhythm defining the physiological state of the individual. The means 40 determines the difference between the values of the measured physiological parameters and the values of the desired physiological parameters. The means 50 determines the physiological parameters associated with an intermediate physiological state EPi between the physiological rhythm of the individual as measured (EP0 or EPt) and the desired final physiological rhythm EPF. The means 30, 40, 50 are, for example, implemented by software means; they are used essentially to carry out steps E4.

The means 60 produces, from the selected intermediate physiological state, a set of stimuli defined by a set of stimulation parameters (ST1M, ST1N, ST1P, ST2P, ST3N, ST3P, etc. . . . ) and comprising one or more stimuli. The means 60 comprises notably a table in which, with each physiological state defined by physiological parameters, a set of stimuli is associated, which makes it possible to reach the associated physiological state, where each set of stimuli is characterized by a set of stimulation parameters. The implementation of the means 60 depends naturally on the type of stimulus to be produced, audio, text, video, etc. The means 70 emits the stimulus produced earlier in the direction to the individual. Depending on the type of stimulus to be emitted, the means 70 is, for example, a display screen, a speaker, etc. The means 60, 70 are used essentially to carry out steps E3, E5.

The device according to the invention can be improved in the same way as the above-described procedure.

A means 80 may also be provided that selects the appropriate type of stimulus. This means is, for example, of the software type, and it can be more or less complex, depending on the number of available types of stimuli and depending on the selection criteria to be taken into account. The following may notably be taken into account:
the effectiveness of a stimulus in comparison to another,
the initial physiological state EP0 of the individual,
the final physiological state EPf to be reached,
the separation between the initial state and the final state,
the time available to pass from the initial state to the final state,
etc.

A calibration means 90 may also be provided to the device, which is capable of testing the effectiveness of different stimuli and the effectiveness of each parameter of the same stimulus, for a given individual, at a given time. The means 90 is used to carry out step E13, and the result of the calibration is then used by the means 80 to select a stimulus.

A means 100 may also be provided that stores in a table a set of physiological states, each corresponding to one or more psychological states, and each characterized by a set of physiological parameters.

The set of means of the device of the invention is controlled by a control means that is not shown in FIG. 3.

### Applications of the Invention

In general, the invention makes it possible to bring an individual, who is initially in a given physiological state EP0, to a desired physiological state EPf, which is more appropriate for an activity that the individual is going to undertake.

A considered application of the invention is the production of learning devices of the type
of a driving simulator, for example, for a motorized engine, such as a plane, a car, etc.
of a self-instruction device for learning a foreign language, theoretical and/or practical instruction, etc.

In this case, the invention is used to bring the learner into special learning conditions, notably conditions of concentration, stress, etc., to test his/her reactions under special conditions, or to bring him/her into conditions of receptivity that are more effective for new learning.

Another possible application of the invention is the production of games, films, or multimedia applications (films, entertainment software, etc.). In this case, the invention is used to bring the player(s) or the viewers into special conditions of receptivity (for example, a feeling of anguish) at a precise time (for example, at the time of a particularly spectacular scene).

Other applications are also possible in the medical field:
depending on the case, to bring a patient into a state of relaxation, release of tension, hypnosis, in view of a surgery, medical consultation, etc.
synchronization between a patient and a psychotherapist, with a view to improving the effectiveness of a psychological consultation (as in neurolinguistic programming)

Other applications are also considered in the field of sports, for the purpose of:
during physical effort, bringing an individual rapidly into a state of cardiac, respiratory, etc., equilibrium
promoting release of tension, individual relaxation of an individual.

Other applications, finally, are considered in the field of professions, such as, for example, those where it is desired to bring several persons temporarily and simultaneously into the same physiological state (which may evolve or not be defined clearly) or into similar physiological states, in order to facilitate the team work of these persons.

1. Procedure to bring an individual from an initial physiological state (EP0) to a final physiological state (EPf), where the physiological state of the individual is defined by a set of physiological parameters (RP1A, RP1B, RP1C, RP1D, RP2A, RP2B, RP3A, RP3B, RP3C, . . . ), which are characteristic of one or more physiological rhythms of the individual, where the set of physiological parameters comprises at least two physiological parameters among a value (RP1A), a variability (RP1B), an amplitude of the variations (RP1C), and a periodicity of the variations (RP1D) of a physiological rhythm (RP1) of the individual, where the procedure comprises the following steps:
E2: determination of the initial physiological state of the individual (EP0),
E3: exposure of the individual for a first period (T1) to a set of stimuli (ST1, ST2, . . . ), comprising at least one stimulus (ST1), where said stimulus is characterized by at least one stimulation parameter (STM1, STN1 or STP1) whose value is a function of the initial physiological state (EP0),
E4: determination of an intermediate physiological state (EPi) between the initial physiological state (EP0) and the final physiological state (EPf),
E5: modification of at least one parameter (STM1) of at least one stimulus (ST 1) of the set of stimuli (ST1, ST2, . . . ) as a function of the intermediate state (EPi), an exposure of the individual to the modified set of stimuli (ST1, ST2, . . . ),
Q8: if the intermediate physiological state (EPi) is different from the desired final physiological state (EPf), repetition of steps E4 and E5.

2. Procedure according to claim 1, comprising also the following steps, which are carried out between steps E5 and Q8:
E6: determination of the physiological state reached by the individual (EPt+1),
Q7: if the physiological state reached by the individual (Ept+1) is different from the intermediate physiological state (EPi), repetition of steps E5 and E6.

3. Procedure according to one of claims 1 or 2, comprising also the following step, which is carried out between steps E6 and E7:
Q9: if the physiological state reached by the individual (Ept+1) is further removed from the intermediate physiological state than the physiological state that was reached previously by the individual (EPt) and determined during step E2 or during a preceding step E6, repetition of steps E3 and following while choosing a new intermediate physiological state (EPi) that is closer to the physiological state reached by the individual (EPt+1).

4. Procedure according to one of claims 1-3, comprising also the following step, which is carried out before step E3:
E10: selection of a set of stimuli, as a function of the initial physiological state (EP0) of the individual, and of the final physiological state (EPf).

5. Procedure according to claim 4, comprising also the following step, which is carried out after step E6:
Q11: if the set of stimuli is not effective or of low effectiveness, carrying out a step E10, then steps E3 and following.

6. Procedure according to one of claims 1-5, comprising also a step of initialization, which comprises the following, substeps, during which:
E0: a physiological state is associated in a table with each desired psychological state, where each physiological state is characterized by a set of physiological parameters (RP1A, RP1B, RP1C, RP1D, RP2A, RP2B, RP3A, RP3B, RP3C, . . . ),
E1: selecting from the table a final physiological state (EPf) as a function of a desired psychological state.

7. Device intended for the production of stimuli that are intended to lead an individual from an initial physiological state (EP0) to a final physiological state (EPf), comprising:
a means (10) of measuring the current physiological state (EP0, EPt) of an individual,
a means (30, 40) of analyzing and comparing the measured physiological state (EP0, EPt) with a reference physiological state (EPf, EPi), which is either an intermediate physiological state (EPi) or a final physiological state to be reached (EPf),
a means (50) of determining the intermediate physiological state (EPi), as a function of the determined current physiological state (EP0, EPt) of the individual and of the final physiological state (EPf) to be reached,
a means (60) of producing a set of stimuli, which comprises at least one stimulus of which at least one stimulation parameter (M, N and/or P) is variable as a function of the determined intermediate state (EPi), and
a means (70) of exposing the individual to the produced set of stimuli.

8. Device according to claim 7, in which the physiological state of the individual is defined by a set of physiological parameters (RP1A, RP1B, RP1C, RP1D, RP2A, RP2B, RP3A, RP3B, RP3C, . . . ), which are characteristic of one or more physiological rhythms of the individual, where the set of physiological parameters comprises at least two physiological parameters, and in which the means (30, 40) of analysis and of comparison comprises:
a means (30) of analyzing the measured current physiological state (EP0, EPt), to extract from it the physiological parameters,
a means (40) of comparing the extracted physiological parameters with corresponding parameters of the reference physiological state.

9. Device according to claim 8, in which the set of physiological parameters comprising at least two parameters among a value (A), a variability (B), an amplitude of the variations (C), and a periodicity of the variations (D) of a physiological rhythm (RP1) of the individual.

10. Device according to one of claims 7-9, in which the measurement means (10) comprises a sensor for measuring a cardiac rhythm, a respiratory rhythm and/or brain waves of the individual.

11. Device according to one of claims 7-10, containing also a calibration means (80) for testing several sets of stimuli and classifying the tested sets of stimuli, as a function of the effectiveness on the physiological state of the individual.

12. Device according to claim 8, comprising also a means (90) of selecting an effective set of stimuli, as a function of the current physiological state (EPt) of the individual.

13. Use of a procedure according to one of claims 1-6, or of a device according to one of claims 7-12, to produce a learning device or a driving simulator.
